(19) United States
(12) Patent Application Publication
(10) Pub. No.: US 2022/0122740 A1
(43) Pub. Date: Apr. 21, 2022

(54) EARLY DETECTION OF INFECTIOUS RESPIRATORY DISEASES

(71) Applicant: SAMSUNG ELECTRONICS CO., LTD., Gyeonggi-do (KR)

(72) Inventors: Ebrahim Nematihosseinabadi, Mountain View, CA (US); Md M. Rahman, San Jose, CA (US); Viswam Nathan, Santa Clara, CA (US); Korosh Vatanparvar, Santa Clara, CA (US); Tousif Ahmed, San Jose, CA (US); Jilong Kuang, San Jose, CA (US); Jun Gao, Menlo Park, CA (US)

(21) Appl. No.: 17/071,763

(22) Filed: Oct. 15, 2020

(51) Int. Cl. G16H 50/80; G16H 40/67; G16H 50/70; G16H 10/60; G16H 50/20; G16H 50/30; G16H 10/20; H04L 29/08; H04W 4/029; A61B 5/08; A61B 5/1455

(52) U.S. Cl. CPC G16H 50/80; G16H 40/67; G16H 50/70; G16H 10/60; G16H 50/20; G06Q 50/265; G16H 10/20; H04L 67/22; H04W 4/029; A61B 5/0823; A61B 5/14551; G16H 50/30

(57) ABSTRACT

Early detection of infectious respiratory diseases includes monitoring, during a predetermined time interval, electronic communications generated by network-connected portable devices, each portable device used by one of a plurality of users. The electronic communications are processed by extracting from each electronic communication biomarkers obtained by processing signals captured by the portable devices, the signals generated in response to sensor-detected respiratory activity of the portable device users. Clusters of users are generated based on the biomarkers and are conditioned based on pre-condition data extracted from the electronic communications, the pre-condition data corresponding to users determined to have pre-existing health conditions. Based on context data extracted from the electronic communications, user co-locations and pre-symptomatic biomarker variation co-occurrences are determined for users who exhibit pre-symptomatic biomarker variations associated with an infectious respiratory disease. An outbreak of the infectious respiratory disease is predicted along with the likelihood of infection for individuals based on the user co-locations and pre-symptomatic biomarker variation co-occurrences.

### TECHNICAL FIELD

This disclosure relates to early detection of outbreaks of infectious respiratory diseases, and more particularly, to determination of the likelihood of infection of individuals and likely patterns and flows of the spread of infectious respiratory diseases.

### BACKGROUND

Epidemiological studies by the U.S. Center for Disease Control and Prevention and other researchers attribute thousands of deaths in the U.S. and millions worldwide to outbreaks of influenza and other respiratory diseases. These diseases not only cause great human suffering but also impose immense economic burdens on communities wherever outbreaks occur. The rapid spread of COVID-19 dramatically highlights the public health challenge of detecting and predicting the course of outbreaks of infectious respiratory diseases.

### SUMMARY

In one or more embodiments, a method includes monitoring during a predetermined time interval electronic communications generated by an ensemble of network-connected portable devices. Each electronic communication corresponds to one of a plurality of users using a corresponding one of the portable devices. The method includes processing the electronic communications by extracting from each electronic communication one or more biomarkers obtained by processing signals captured by a corresponding portable device, the signals generated in response to sensor-detected respiratory activity of a user of the corresponding portable device. The method includes generating one or more clusters of users based on the one or more biomarkers corresponding to each of the users, and conditioning the one or more clusters based on pre-condition data extracted from the electronic communications, the pre-condition data corresponding to users who are determined to have a pre-existing health condition. The method includes determining, based on context data extracted from the electronic communications, user co-locations and pre-symptomatic biomarker variation co-occurrences among a pool of users who exhibit pre-symptomatic biomarker variations associated with an infectious respiratory disease. The method includes predicting a location of a possible outbreak of the infectious respiratory disease based on the user co-locations and pre-symptomatic biomarker variation co-occurrences.

In one or more embodiments, a system includes a processor configured to initiate operations. The operations include monitoring during a predetermined time interval electronic communications generated by an ensemble of network-connected portable devices. Each electronic communication corresponds to one of a plurality of users using a corresponding one of the portable devices. The operations include processing the electronic communications by extracting from each electronic communication one or more biomarkers obtained by processing signals captured by a corresponding portable device, the signals generated in response to sensor-detected respiratory activity of a user of the corresponding portable device. The operations include generating one or more clusters of users based on the one or more biomarkers corresponding to each of the users, and conditioning the one or more clusters based on pre-condition data extracted from the electronic communications, the pre-condition data corresponding to users who are determined to have a pre-existing health condition. The operations include determining, based on context data extracted from the electronic communications, user co-locations and pre-symptomatic biomarker variation co-occurrences among a pool of users who exhibit pre-symptomatic biomarker variations associated with an infectious respiratory disease. The operations include predicting a location of a possible outbreak of the infectious respiratory disease based on the user co-locations and pre-symptomatic biomarker variation co-occurrences.

In one or more embodiments, a computer program product includes a computer readable storage medium having instructions stored thereon. The instructions are executable by a processor to initiate operations. The operations include monitoring during a predetermined time interval electronic communications generated by an ensemble of network-connected portable devices. Each electronic communication corresponds to one of a plurality of users using a corresponding one of the portable devices. The operations include processing the electronic communications by extracting from each electronic communication one or more biomarkers obtained by processing signals captured by a corresponding portable device, the signals generated in response to sensor-detected respiratory activity of a user of the corresponding portable device. The operations include generating one or more clusters of users based on the one or more biomarkers corresponding to each of the users, and conditioning the one or more clusters based on pre-condition data extracted from the electronic communications, the pre-condition data corresponding to users who are determined to have a pre-existing health condition. The operations include determining, based on context data extracted from the electronic communications, user co-locations and pre-symptomatic biomarker variation co-occurrences among a pool of users who exhibit pre-symptomatic biomarker variations associated with an infectious respiratory disease. The operations include predicting a location of a possible outbreak of the infectious respiratory disease based on the user co-locations and pre-symptomatic biomarker variation co-occurrences.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

The inventive arrangements are illustrated by way of example in the accompanying drawings. The drawings, however, should not be construed to be limiting of the inventive arrangements to only the particular implementations shown. Various aspects and advantages will become apparent upon review of the following detailed description and upon reference to the drawings.

FIG. 1 illustrates an example networking environment in which an infectious respiratory disease detection system is operative in accordance with one or more embodiments described herein.

FIG. 2 illustrates an example infectious respiratory disease detection system in accordance with one or more embodiments described herein.

FIG. 3 is a flowchart of an example method for early detection of an infectious respiratory disease outbreak in accordance with one or more embodiments described herein.

FIG. 4 is a flowchart of an example method of confirming with active testing a likely infectious respiratory disease outbreak in accordance with one or more embodiments.

FIG. 5 is a flowchart of an example method of contact tracing in accordance with one or more embodiments.

FIG. 6 illustrates an example computing node in which an infectious respiratory disease detection system can be implemented in accordance with one or more embodiments described herein.

FIG. 7 illustrates an example portable device that as one among an ensemble of portable devices can be used with an infectious respiratory disease detection system in accordance with one or more embodiments described herein.

### DETAILED DESCRIPTION

While the disclosure concludes with claims defining novel features, it is believed that the various features described within this disclosure will be better understood from a consideration of the description in conjunction with the drawings. The process(es), machine(s), manufacture(s) and any variations thereof described herein are provided for purposes of illustration. Specific structural and functional details described within this disclosure are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates to early detection of outbreaks of infectious respiratory diseases, and more particularly, to determination of the likelihood of infection of individuals and likely patterns and flows of the spread of infectious respiratory diseases. Effective containment of a respiratory disease outbreak usually depends on early detection of respiratory disease infections within a given population. Compiling data on infections from individuals, their doctors, and/or hospitals may take two weeks or more before public health officials are able to adequately discern an outbreak. Data derived from search queries, social media, tweets, blogs, and the like (so-called “infodemiology”) may be compiled more quickly but, at best, is merely indirect, anecdotal evidence of a possible outbreak. Infectious respiratory diseases, like many other types of infectious diseases, can be highly contagious. As such, delay in acquiring reliable, direct evidence of an infectious respiratory disease outbreak can significantly impede efforts to contain the outbreak.

The systems, methods, and computer program products disclosed herein detect a possible infectious respiratory disease outbreak and predict the course of the disease over time. The probability of an infectious respiratory disease outbreak and predictions of the pattern and flow of the spread of the disease are based on real time and near-real time data. The data is derived from sensor-detected signals generated in response to physiological activities and conditions of multiple individuals. The data can be passively acquired from an ensemble of portable devices (e.g., smartphone, smartwatch, earbuds).

An inventive aspect of the embodiments disclosed herein is the clustering of a population (e.g., portable device users) into groups (clusters) based on biomarkers extracted from data transmitted by multiple devices (e.g., wirelessly from the ensemble of portable devices), the biomarkers corresponding to specific device users. As defined herein, a “biomarker” is a value, measure, or other quantifiable indicator of an individual's physiology, including the condition or state of one or more of the individual's body organs or the functioning thereof. For example, an individual's breathing cycle (time of one repetition of the three-fold pattern of breathing air into the lungs (inhalation), oxygen-carbon dioxide exchange, and breathing air out of the lungs (exhalation)) is a biomarker. The individual's respiratory rate (typically measured as breaths per minute (BPM)) is a biomarker. The individual's blood pressure, heart rate, and heart rate variability, are biomarkers, as is a measure of the individual's peripheral capillary oxygen saturation (SpO2).

Clustering can separate individuals (e.g., portable device users) who exhibit healthy biomarkers from ones who exhibit one or more pre-symptomatic biomarker variations associated with an infectious respiratory disease. As defined herein, a “biomarker variation” is a biomarker that deviates by more than a predetermined amount from a predetermined norm or that changes over a predefined time interval by more than a predetermined amount. For example, a respiratory rate that is more than two standard deviations above or below a mean or median respiratory rate can constitute a biomarker variation. A respiratory rate that increases by more than eight BPM within a three-day period, for example, can constitute a biomarker variation. As defined herein, a “pre-symptomatic biomarker variation” is one that occurs before a symptom that enables a diagnosis or is assistive in the diagnosis of a disease.

Another inventive aspect of the embodiments disclosed herein is the conditioning of clusters. A cluster may comprise healthy individuals who, by virtue of an as-yet undiagnosed respiratory disease infection, exhibit the same pre-symptomatic biomarker variation exhibited by one or more others in the cluster who have a pre-existing health condition but are not infected. Conditioning the cluster disentangles the infected individuals from those merely exhibiting symptoms due to a pre-existing health condition. Through the process of conditioning the initially generated clusters, a pool of users who exhibit one or more pre-symptomatic biomarker variations likely due to a respiratory disease infection can be identified.

Exhibition of pre-symptomatic biomarker variations associated with an infectious respiratory disease by multiple individuals, however, may be merely a random occurrence. The co-occurrence of pre-symptomatic biomarker variations exhibited by co-located individuals—especially in large numbers—makes it more likely that the pre-symptomatic biomarker variations are due to an outbreak of an infectious respiratory disease associated with the specific pre-symptomatic biomarker variations. Co-location corresponds to two or more individuals being within the same predefined geographic area to cause a potential contagious contact, “contagious contact” being defined as human-to-human interaction (direct or at a distance) that, depending on the nature of the specific infectious disease, enables transmission of the disease from an infected individual to another individual. For example, with respect to an infectious respiratory disease, two individuals located less than six feet from one another may constitute a contagious contact. In some embodiments, co-location is predefined as any distance that renders the probability of transmission of an infectious respiratory disease greater than a predetermined level of probability (e.g., 50 percent or greater).

Accordingly, the systems, methods, and computer program products disclosed herein are able to predict location(s) of a possible outbreak of the infectious respiratory disease based on the user co-locations and pre-symptomatic biomarker variation co-occurrences. The co-location(s) and co-occurrences are determined based on conditioned clustering of individuals. The individuals can be users of portable devices (e.g., smartphones, smartwatches, earbuds) and the individuals' biomarkers can be passively obtained from the individuals' portable devices. Each portable device can be configured to extract one or more biomarkers from signals generated by the portable device's sensors (e.g., IMUs, PPGs) in response to physiological activity of a user of the portable device.

Further inventive aspects of the embodiments described within this disclosure are described in greater detail with reference to the figures below. For purposes of simplicity and clarity of illustration, elements shown in the figures have not necessarily been drawn to scale. For example, the dimensions of some of the elements may be exaggerated relative to other elements for clarity. Where considered appropriate, reference numbers are repeated among the figures to indicate corresponding, analogous, or like features.

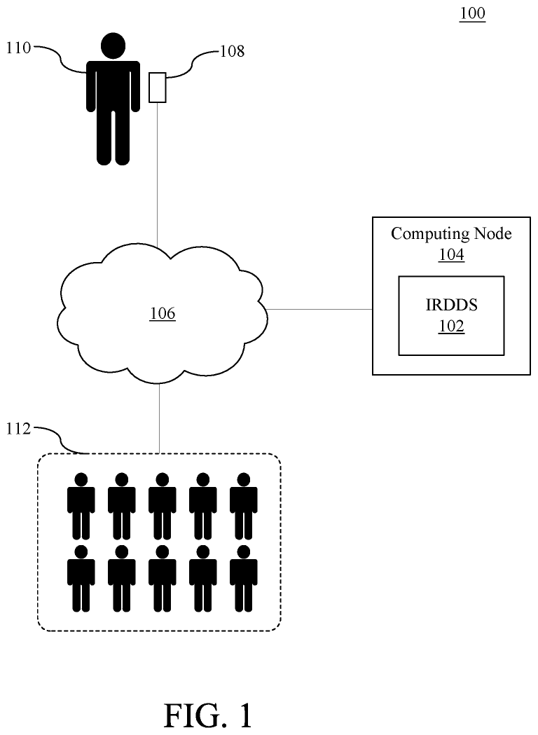

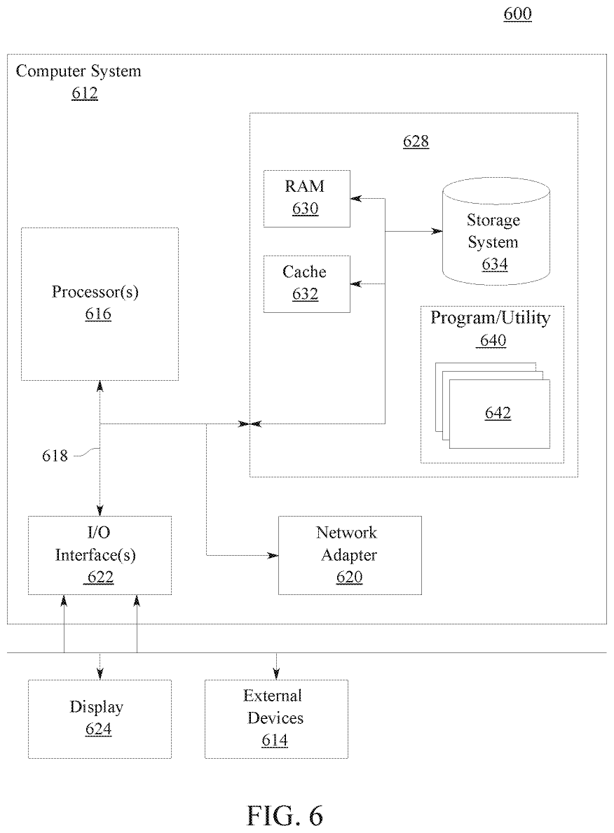

FIG. 1 depicts networking environment 100. Networking environment 100 is an example networking environment in which infectious respiratory disease detection system (IRDDS) 102, according to one embodiment, is operative. Networking environment 100 illustratively includes computing node 104. Although not explicitly shown, other computing nodes can also be included in networking environment 100. IRDDS 102 is illustratively implemented in computing node 104. Computing node 104 can include any of the various components that comprise computing node 600 described with reference to FIG. 6. Such a computing node can include one or more computer systems (e.g., cloud-based servers), each having one or more processors, memory, and various other of the components illustrated by computer system 612 in FIG. 6. Illustratively, IRDDS 102 is implemented in software that is stored in a memory and executes on one or more processors of one or more computer systems of computing node 104. In other embodiments, IRDDS 102 can be implemented in dedicated circuitry or a combination of software and circuitry.

Networking environment 100 illustratively includes communications network 106 configured to facilitate wireless and/or wired connections between computing node 104 and various networked computing and communication devices. In some embodiments, communications network 106 is an integration of distinct networks such as a cellular phone network operatively coupled with a cloud-based computing network. IRDDS 102, operating on computing node 104, is communicatively coupled via communications network 106 to portable device 108 of user 110. IRDDS 102 can communicatively couple to various other portable devices (not explicitly shown), which are used by plurality of users 112.

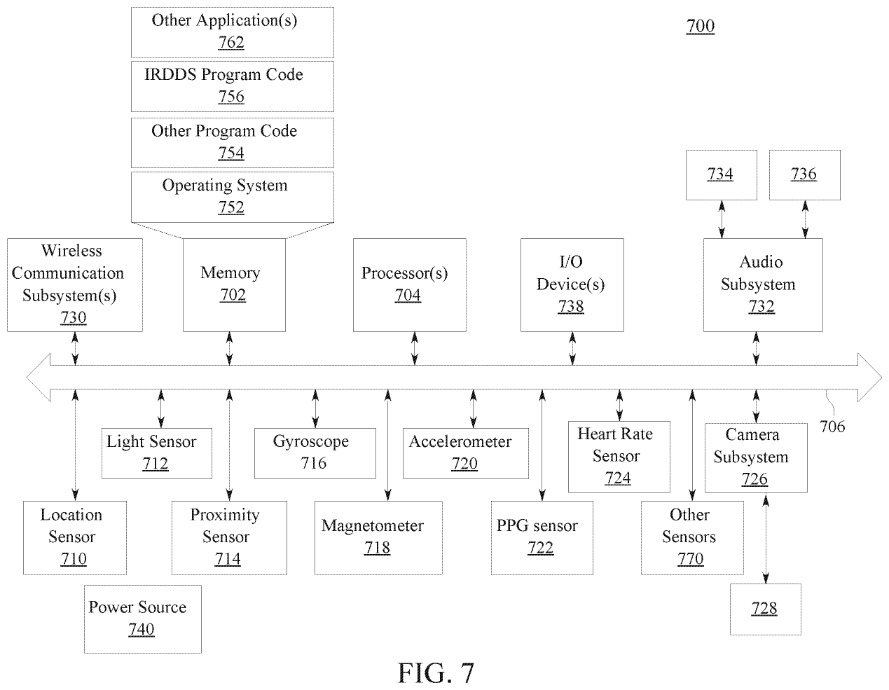

Portable device 108, as well as ones used by plurality of users 112, can be a smartphone, smartwatch, microphone-enhanced earbuds, or similar such portable device that can be comfortably carried or worn by the users. The portable devices can be used singly or in conjunction with another computing or communication device and/or one or more peripheral devices (e.g., smartphone communicatively coupled to a smartwatch and/or earbuds). An example portable device is portable device 700, whose components are described in detail with reference to FIG. 7. The components include one or more processors, memory, and various sensors. The sensors can include, for example, inertial measurement units (IMUS) (e.g., triaxial accelerometer, triaxial gyroscope, triaxial magnetometer), photoplethysmogram (PPG) sensors, heart rate monitors, blood pressure monitors, and the like.

The sensors generate signals in response to a user's physiological activity. For example, the IMUs of a smartphone or smartwatch held against a user's chest can generate signals in response to the expansion and contraction of the user's ribcage during a breathing cycle. A smartphone microphone or one embedded in an earbud can capture acoustic signals generated by the user's coughing or wheezing. A PPG sensor can measure SpO2 of the user. Other portable device sensors can detect physiological attributes of the user such as level of physical activity and sleep patterns.

Different biomarkers can be extracted from the signals generated by the sensors in response to the user's physiological activity. For example, based on signals generated by a smartphone or smartwatch IMU, respiratory biomarkers such as respiratory rate or lung functioning (e.g., FEV1, FVC, FEV1/FVC) can be obtained. Other biomarkers can include the user's respiratory rate, heart rate, heart rate variability, blood pressure, SpO2, and the like.

Portable device 108 illustratively operates as part of an ensemble of portable devices (not explicitly shown) that are used by other users 112 and that, performing in like manner, extract biomarkers from sensor-detected signals generated in response to physiological activity of the respective users. User 110 and other users 112 can agree in advance to each convey via communications network 106 electronic communications containing the users' extracted biomarkers. The electronic communications can be generated automatically in response to each portable device passively monitoring the physiological activity of a corresponding user by extracting biomarkers from sensor-generated signals in response to the physiological activity. The voluntary sharing of the information in electronic communications corresponding to multiple users enables IRDDS 102 to provide each of the users and/or public health officials with an early warning of a possible infectious respiratory disease outbreak determined based on an aggregation of the jointly shared users' data.

IRDDS 102 can continuously or intermittently monitor the electronic communications generated by the ensemble of network-connected portable devices. IRDDS 102 can use a sliding window to determine information based on data collected over a predetermined time interval (e.g., every two to three days). IRDDS 102 extracts biomarkers from the electronic communications and combines the biomarkers with context data extracted from the electronic communications, the context data pertaining to possible pre-existing health conditions of some of the users. Pre-conditions can include, for example, chronic lung disease, chronic kidney disease, heart disease, liver disease, immunocompromise, diabetes, obesity, age-related factors, and the like.

IRDDS 102 for a population of users whose electronic communications from the ensemble of portable devices are monitored generates clusters comprising sub-populations, or groups, of the population of portable device users. Each cluster can be generated to include users whose biomarkers indicate statistically similar health characteristics. The clusters are conditioned by the pre-determined health data to further refine the clusters, for example, based on identifying users affected by one or more pre-existing health conditions. IRDDS 102 further extracts location data from the electronic communications to determine co-locations and co-occurrences of pre-symptomatic biomarker variations among the population of users. Based on location information, IRDDS 102 identifies a growth pattern within the selected clusters. The growth pattern can reveal a statistically significant increase in co-located users who experienced or are experiencing co-occurrences of pre-symptomatic biomarker variation indicative of an emerging outbreak of an infectious respiratory disease. IRDDS 102, based on data collected during a predetermined time interval, can determine how rapidly the number of users who experience pre-symptomatic biomarker variations is increasing. A rapid rise (e.g., exponential increase) in the number of individuals who exhibit one or more pre-symptomatic biomarker variations is an indication of an infectious respiratory disease outbreak.

Those individuals who, although otherwise free of a pre-existing condition, exhibit one or more pre-symptomatic biomarker variations and who are co-located within a region showing a marked increase in the number of individuals exhibiting pre-symptomatic biomarker variations are identified by IRDDS 102 as individuals likely infected by the infectious respiratory disease. A marked increase can be a sharp rise in the number of individuals within a predetermined time interval (e.g., six days to two weeks). An exponential rise in the number, for example, can indicate a high probability of an infectious respiratory disease outbreak.

In an example scenario, a cluster may comprise a pool of users who exhibit one or more pre-symptomatic biomarker variations associated with an infectious respiratory disease. The pre-symptomatic biomarker variations can include, for example, changes in the users' respiratory rates (e.g., above-normal elevation), increased heart rate and/or heart rate variability, decreased SpO2, increased lung function (e.g., FEV1, FVC, FEV1/FVI) variability, mild breathlessness indicated by increased pause time during speech, changes in vocal characteristics (e.g., tone, shimmer, jitter), and/or increased cough frequency. Some pre-symptomatic biomarker variations can be due to a factor other than the onset of an infectious respiratory disease, such as a pre-existing health condition (e.g., COPD, asthma, gastroesophageal reflux).

Cluster conditioning, however, can reveal whether a statistically significant number of members in the cluster are not afflicted by a pre-existing health condition, thus suggesting a possible infectious respiratory disease outbreak. Different individuals with different health backgrounds and pre-conditions may exhibit different levels of variations in their biomarkers. Accordingly, the different individuals can cause different clusters to be generated according to the clustering procedure disclosed herein. Cluster conditioning initially identifies the dominant pre-condition of individuals in each of the clusters. Naturally and usually the dominant cluster is the one related to healthy individuals, given that a majority of individuals are typically healthy. In addition to the dominant cluster of healthy individuals, other smaller clusters are included in the next stage of assessment if individuals in each of the smaller clusters are revealing the infection with the respiratory disease as well. Therefore, cluster conditioning can include those clusters as well if they are dominantly populated with a specific pre-condition. If a cluster is populated predominantly with a pre-condition such as asthma, diabetes, or other pre-existing health condition, then that whole cluster will be sent to the next stage in addition to the dominant cluster for context-based assessment (co-location, co-occurrence).

A spatial sweep of the spatial data—that is, identifying distinct locations of each member of the cluster—can reveal that a significant number of the members were co-located within geographical distances to have contagious contact with at least one other member. The data can be input into a growth pattern model to determine whether an increase in the number of members of the pool indicates an onset of an infectious respiratory disease. For example, as noted above, exponential growth in the number during the predetermined time interval can indicate the onset of an infectious respiratory disease. Based on the growth pattern model, likely timing of the spread of the infectious respiratory disease can be predicted.

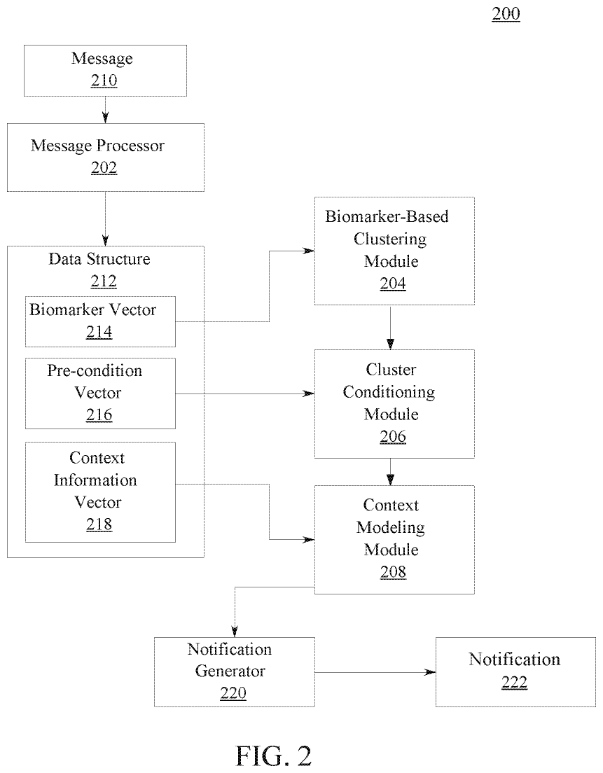

FIG. 2 depicts in greater detail individual components of example IRDDS 200 according to one embodiment. Illustratively, IRDDS 200 includes message processor 202, biomarker-based clustering module 204, cluster conditioning module 206, and context modeling module 208. IRDDS 200 monitors electronic communications generated by an ensemble of network-connected portable devices, each electronic communication including one or more respiratory biomarkers extracted by processing signals captured by a corresponding one of the portable devices. The signals, as described above, are generated in response to sensor-detected respiratory activity of users of the corresponding portable devices.

Example message 210, among the multiple messages monitored by IRDDS 200, is processed by message processor 202. Message processor 202 generates data structure 212 by processing example message 210. Data structure 212 comprises biomarker vector 214, pre-condition vector 216, and context information vector 218. Message processor 202 generates the data structure for each monitored message of portable device users, and extracts biomarker, pre-condition, and context vectors from the data structures. Message processor 202 extracts biomarker vector 214 from data structure 212 and configures the vector as a feature vector that is input into biomarker-based clustering module 204. Biomarker-based clustering module 204, generates one or more clusters from the input of multiple biomarker vectors configured as feature vectors. Each cluster comprising a distinct group of users from among the total population of users of the ensemble of portable devices, the clustering based on the specific biomarkers corresponding to each of the users.

In certain embodiments, biomarker-based clustering module 204 implements one or more similarity-based clustering procedures (e.g., K-means clustering) to group the population of portable device users into distinct clusters based on biomarkers extracted by each user's portable device. The clustering procedures partition observations (a set of biomarkers extracted from messages corresponding to a specific individual) into distinct clusters such that observations within a cluster are similar to one another based on a predetermined distance metric (e.g., a distance matrix or similarity/dissimilarity matrix), while observations in different clusters are not similar. Such procedures can be used for unsupervised learning for which no correctly labeled set of training observations is available but with which there is assumed to be some degree of heterogeneity among the population of observations. By implementing a clustering technique, biomarker-based clustering module 204 can discover structure—that is, distinct clusters—within the population, the structure determined by the biomarkers associated with each member of the population, namely, the portable device user's whose messages are monitored by IRDDS 200.

In some embodiments, biomarker-based clustering module 204 implements K-means clustering. Underlying the procedure is the notion that a reliable clustering of observations is the one that minimizes within-cluster variation, the variation measured by pairwise squared Euclidean distances between observations within each cluster. Thus, biomarker-based clustering module 204 can perform clustering based on users' biomarkers by framing the task as an optimization problem in which, over all K clusters, the sums of the pairwise squared Euclidean distances between observations within each cluster are minimized. In other embodiments, biomarker-based clustering module 204 can implement other clustering procedures, including K-median clustering and fuzzy c-mean clustering. In still other embodiments, biomarker-based clustering module 204 can implement other unsupervised and/or statistical learning procedures.

IRDDS 200 monitors electronic communications generated by an ensemble of network-connected portable devices. Messages can be monitored over extended periods. A predetermined time interval (e.g., two to three days) can operate as a sliding window during which pre-symptomatic variations in users' biomarkers can be identified and clusters generated according to the pre-symptomatic variations. A cluster generated by biomarker-based clustering module 204 can group users who exhibit pre-symptomatic biomarker variations that indicate, for example, adverse changes in the users' respiratory rates or lung functioning (e.g., FEV1, FVC, FEV1/FVI). Other pre-symptomatic biomarker variations can be increased heart rate and/or heart rate variability, decreased SpO2, and/or increased cough frequency.

The clusters generated by biomarker-based clustering module 204 are conditioned by cluster conditioning module 206. Cluster conditioning module 206 conditions the clusters based on pre-condition vector 216 extracted from example message 210, as well as from other pre-condition vectors extracted from other electronic communications, by message processor 202. Pre-condition vectors reveal certain users' underlying pre-existing health conditions. Pre-conditioning the clusters based on data gleaned from the pre-condition vectors disentangles pre-symptomatic variations in users' biomarkers that may be due to the onset of a respiratory infection from biomarker variations that are due to one or more users' pre-existing health condition. Conditioning the clusters generated by biomarker-based clustering module 204 reveals a pool of users who exhibit one or more pre-symptomatic biomarker variations potentially associated with an infectious respiratory disease and not due to a pre-existing health condition. A key to determining whether pre-symptomatic biomarker variations are merely random events or, instead, indicate a possible outbreak of an infectious respiratory disease is to assess the co-occurrences of the users' pre-symptomatic biomarker variations and the users' closeness to one another (co-location), which determines the contagious contacts occurring during a specific time interval.

Message processor 202 extracts context data from data structure 212 corresponding to example message 210. Message processor 202 generates context information vector 218, as well as context information vectors from other electronic communications of other users identified in the pool of users who exhibit one or more pre-symptomatic biomarker variations. Each context vector comprises spatial data corresponding to a user of a corresponding portable device monitored by IRDDS 200. The spatial data indicates the location at which a user who is experiencing or experienced one or more pre-symptomatic biomarker variations is located. Each context vector can include temporal data. Based on context data, context modeling module 208 determines user co-locations and pre-symptomatic biomarker variation co-occurrences among a pool of users who exhibit pre-symptomatic biomarker variations associated with an infectious respiratory disease. The user co-locations and pre-symptomatic biomarker variation co-occurrences provide input to a model for determining whether a community is likely to experience an outbreak of an infectious respiratory disease. For example, exponential growth in the number of users experiencing pre-symptomatic biomarker variations can be an indication of the onset of a respiratory disease infection. A rapid rise in the number during a predetermined time interval that is especially brief is an especially significant indication.

Context modeling module 208, based on the determined user co-locations and pre-symptomatic biomarker variation co-occurrences can predict a location of a possible outbreak of the infectious respiratory disease. Context modeling module 208, additionally, can predict the timing of the outbreak. Optionally, notification generator 220 responds to a prediction of an outbreak and generates electronic notification 222 and conveys the electronic notification via a network connection to a communication device of one or more individuals. The electronic notification can indicate the predicted location of the outbreak. The notification, optionally, indicates a predicted timing of the outbreak of the infectious respiratory disease. Interested individuals can include the users themselves who might otherwise not be aware of a danger of infection merely from experiencing pre-symptomatic biomarker variations. Almost certainly the users would otherwise be unaware that a rapid increase in the number other users similarly experiencing pre-symptomatic biomarker variations is occurring and that the rapid increase indicates a likely outbreak of the infectious respiratory disease. Notification also can alert public health officials before other indicators are available.

In some embodiments, IRDDS 200 further estimates additional epidemiological parameters (e.g., epidemiological model parameters) that can be used to advise users and/or public health officials on additional aspects of an outbreak of an infectious respiratory disease. Context modeling module 208 estimates various epidemiological parameters based on the context data (locations and times) corresponding to the pool of users who exhibit one or more pre-symptomatic biomarker variations associated with an infectious respiratory disease. The epidemiological parameters estimated using the data provide input into one or more models for predicting an infectious respiratory disease outbreak, as well as the rapidity and pattern in which the disease can be expected to spread, including a likely next “hotspot” of respiratory infections. Context modeling module 208, in certain embodiments, implements one or more of epidemiological models. One class of models implemented by context modeling module 208 comprises compartmental models in which members of a population are assigned to labeled compartments and individuals in the population move between compartments over the course of an infectious disease outbreak or epidemic. The compartments in the SIR model, for example, are labeled S for “susceptible,” I for “infectious,” and R for “recovered” or “resistant” or “removed” (deceased). A similar model is the SEIS model, comprising compartments labeled S for “susceptible,” E for “exposed,” I for “infectious,” and S for “susceptible” (again).

With the SIR model, S is the number of susceptible individuals. I is the number of infectious individuals. If an individual in compartment S comes into contact with an infectious individual (e.g., contagious contact), the former individual transitions to compartment I, the pool of individuals infected and capable of infecting others. R is the number of infected individuals who have recovered or died. In different versions, the SIR model can be constructed as a set of ordinary differential equations or a set of stochastic processes. The following set of first-order ordinary differential equations is one example:

ds(t)/dt=−bs(t)i(t)

di(t)/dt=bs(t)i(t)−ki(t)

dr(t)/dt=ki(t),

where the first equation is the rate of change in the number of susceptible individuals, the second equation is the rate of change in the number of infected individuals, and the third equation is the rate of change in recovered individuals. The system is solved, under specified initial conditions, for the model parameters b and k.

Among the epidemiological parameters estimated by context modeling module 208 using the various models is basic reproduction number, R₀, the expected number of infections directly generated by a single individual's transmission of the disease in a population in which all individuals are susceptible to infection. Another epidemiological parameter estimated is growth rate, α, the rate at which the number of infections increases in a specific location. Another epidemiological parameter is generation time, G, the average time between when an individual is infected and when the individual infects others. Generation time, G, can be calculated as an average of the individual generation times of individuals in the pool of users who exhibit pre-symptomatic biomarker variations, or determined more accurately, through contact tracing described in detail below. Reproduction number, R₀, can be determined based on the following equation:

R₀=1+α(τ_{E}+τ_{I})+α²τ_{E}τ_{I},

where α is the growth rate, τ_{E} is the average duration of an individual's exposed state, and τ_{I} is the average duration of an individual's infectious state.

Using growth rate, α, context modeling module 208 provides an indication of how rapidly an infection is growing at each location. The information can be used by context modeling module 208 to identify “hotspots, locations in which the number of infections is increasing exponentially with a large α value. IRDDS 200 can monitor α values over time for various locations and nearby vicinities (or neighborhoods) to determine the pattern and flow of a spreading infectious disease. Based on the flow and direction, a likely next “hotspot” can be identified by context modeling module 208, which can assist in the deployment of public health resources.

Context modeling module 208 can identify locations where growth rate, α, is negative. This can guide public health officials in deciding when and to shift recourses away from a location. A determination that the growth rate, α, has become negative can also be used in determining appropriate time for removing social distancing restrictions.

An epicenter can be identified by a geographic center of a geographic region in which users included in a conditioned, dominant cluster are located. A user in the same cluster whose pre-symptomatic biomarker variation occurred earliest in time when the number of such users began to increase exponentially can be identified as “patient zero” for the outbreak. These determinations can assist in identifying the origin of the outbreak. The determinations also can assist experts in modeling the progress of the infectious disease progress and can enable public health officials to more efficiently perform contact tracing. Users and other members of an affected community who are suspected of having been infected can be located through contact tracing based on location histories, as well as Bluetooth and Wi-Fi connections with users' permission. Thus, IRDDS 200 can identify not only users who exhibit one or more pre-symptomatic biomarker variations associated with an infectious respiratory disease but also individuals who have had contagious contact with the identified users. Although not identifying all infected and exposed individuals, IRDDS 200 can identify enough such individuals to enable public health officials to initiate contact tracing.

In some embodiments, each portable device among the ensemble of portable devices monitored by IRDDS 200 transmits a message containing one or more biomarkers only under prescribed conditions. For example, each portable device can condition the transmitting of a biomarker-containing message on an initial biomarker anomaly identification. A biomarker anomaly can be defined by a biomarker whose value (e.g., real number), measure, or other quantifiable indicator of a condition or state of a user's physiology is greater than a predetermined distance (e.g., two standard deviations) from a base line (e.g., mean or median of the biomarker). Accordingly, in certain embodiments, IRDDS 200 also includes a biomarker baseline calculator and anomaly identifier, both implemented in software, dedicated circuitry, or a combination thereof. The biomarker baseline calculator can determine a mean or median biomarker level of a user based on a sample of measurements of user-specific biomarkers. For example, the biomarker baseline calculator can obtain a mean from a sample of measured respiratory rates of the user and determine a corresponding sample standard deviation. Anomaly identifier identifies a biomarker anomaly, in some embodiments, in response to detecting that a current measure of the user's respiratory rate is more than two standard deviations above or below the sample mean or median respiratory rate of the user.

The conditioning of transmissions of biomarker-containing messages provides at least three distinct advantages. Firstly, clusters generated based on monitored messages more reliably represent actual epidemiological conditions (e.g., acute infectious conditions). Secondly, conditioning the transmissions effects savings in power consumption by each of the ensemble of portable devices. Thirdly, the data storage demands of the computing node that supports IRDDS 200 are reduced. In alternative embodiments, the baseline of any one of various biomarkers (e.g., the mean or median and corresponding standard deviation) can be derived from clinical data obtained from a plurality of healthy subjects in a clinical setting.

In still other embodiments, IRDDS 200 can include an additional component for enhancing the reliability of predictions and estimates based on the pool of users who exhibit one or more pre-symptomatic biomarker variations associated with an infectious respiratory disease. To have a more reliable user cohort group that is representative of the characteristics of the infectious respiratory disease, each member of the pool of users can be electronically notified via a corresponding portable device (or separate device) and requested to perform one or more active tests. The electronic notification can include instructions for performing the requested active test(s). For example, the users can each be requested to perform a spirometry test using with a smartphone. Each user, for example, can be requested to cough into the microphone of the smartphone or hold the smartphone to the user's chest to sense the flexing of the user's ribcage during breathing cycles. IRDDS 200 electronically collects and analyzes the actively performed tests and uses the result to limit members within the selected cluster to only those who exhibit the same or sufficiently similar symptoms.

In yet other embodiments, IRDDS 200 additionally collects data generated by those devices among the ensemble of portable devices capable of sensing the proximity of a user to other users. For example, Bluetooth or Wi-Fi can be utilized for contact-tracing among the pool of users who exhibit one or more pre-symptomatic biomarker variations associated with an infectious respiratory disease. The collected data can assist public health officials by providing a more efficient and targeted schema for conducting contact tracing. The IRDDS-collected data also can be used to identify early cases of infection and points of contact based on the associated co-locations and co-occurrences of users who exhibited similar biomarkers.

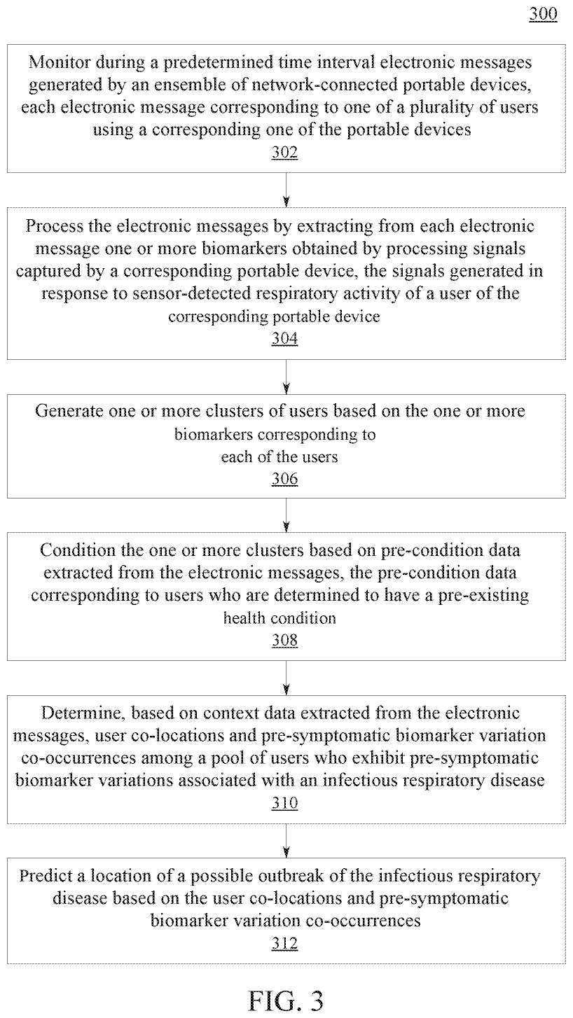

FIG. 3 is a flowchart of example method 300 of early detecting an infectious respiratory disease outbreak in accordance with one or more embodiments. Method 300 can be performed by a system the same or similar to the ones described with reference to FIGS. 1 and 2. The system at block 302 monitors electronic communications generated by an ensemble of network-connected portable devices. Each electronic communication corresponds to one of a plurality of users, each user using a corresponding one of the portable devices. The system can monitor the electronic communications during a predetermined time interval.

At block 304, the system processes the electronic communications by extracting from each electronic communication one or more biomarkers obtained by processing signals captured by a corresponding portable device. The signals are generated in response to sensor-detected respiratory activity of a user of the corresponding portable device.

At block 306, the system generates one or more clusters of users based on the one or more biomarkers corresponding to each of the users, and at block 308 conditions the one or more clusters based on pre-condition data extracted from the electronic communications. The pre-condition data corresponds to users who are determined to have a pre-existing health condition.

The system at block 310 determines, based on context data extracted from the electronic communications, user co-locations and pre-symptomatic biomarker variation co-occurrences among a pool of users who exhibit pre-symptomatic biomarker variations associated with an infectious respiratory disease.

At block 312, the system predicts a location of a possible outbreak of the infectious respiratory disease based on the user co-locations and pre-symptomatic biomarker variation co-occurrences. The system also can predict the timing of the possible outbreak. Optionally, the system can convey an electronic notification via a network connection to a communication device of at least one individual. The electronic notification can indicate the predicted location. The notification can indicate a predicted timing of the possible outbreak of the infectious respiratory disease.

Individuals who, although otherwise free of a pre-existing condition, exhibit one or more pre-symptomatic biomarker variations and who are co-located within a region showing a marked increase in the number of individuals exhibiting pre-symptomatic biomarker variations can be identified by the system as individuals likely infected by the infectious respiratory disease. A marked increase can be a sharp rise in the number of individuals within a predetermined time interval (e.g., six days to two weeks). An exponential rise in the number, as already noted, can indicate a high probability of an infectious respiratory disease outbreak.

In some embodiments, each electronic communication monitored by the system is an electronic communication conveyed over a communications network by a corresponding network-connected portable device only in response to a specified condition. The specified condition can be identification of a biomarker anomaly, whereby the messages are conveyed in response to the corresponding portable device identifying the biomarker anomaly associated with a user of the corresponding device. The biomarker anomaly can be identified based on a clinically determined baseline derived from biomarkers obtained from each of a plurality of healthy subjects. In other embodiments, the biomarker anomaly can be identified based on an individual user baseline, which corresponds to the user of the corresponding portable device.

In still other embodiments, the system in addition to predicting the location of a possible outbreak of the infectious respiratory disease can also estimate one or more epidemiological model parameters. The epidemiological parameters can be based on the context data extracted from users' electronic communications.

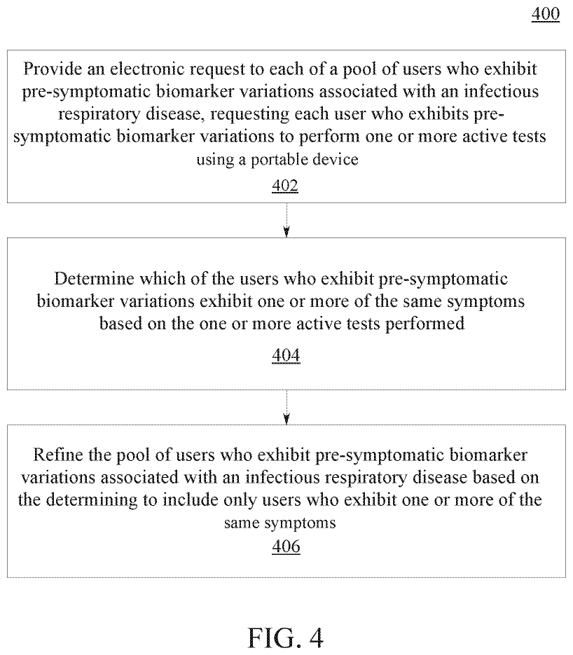

FIG. 4 is a flowchart of example method 400 for confirming with active testing the likely respiratory disease outbreak in accordance with one or more embodiments. Method 400 can be performed by a system the same or similar to the ones described with reference to FIGS. 1 and 2. The system at block 402 provides an electronic request to each of a pool of users who exhibits pre-symptomatic biomarker variations associated with an infectious respiratory disease. The pool of users who exhibit pre-symptomatic biomarker variations associated with an infectious respiratory disease can be determined by extracting biomarkers from sensor-detected signals with the portable devices of the users, as described above. Clusters of the users, based on the biomarkers, can be generated, and the clusters conditioned based on the procedures also described above. The electronic request can be conveyed to each user who exhibits pre-symptomatic biomarker variations requesting that the user perform one or more active tests using a portable device.

At block 404, the system determines which of the users who exhibit pre-symptomatic biomarker variations exhibit one or more of the same symptoms based on the one or more active tests performed. The system at block 406 refines the pool of users who exhibit pre-symptomatic biomarker variations associated with an infectious respiratory disease based on determining which users exhibit one or more of the same symptoms. The system can refine the pool by including only users who exhibit one or more of the same symptoms, while excluding any other users. The refined pool can confirm a predicted outbreak. For example, if the number of users who exhibit one or more of the same symptoms is greater than a predetermined number, the statistical likelihood of an emerging or on-going outbreak of the infections respiratory disease can be confirmed.

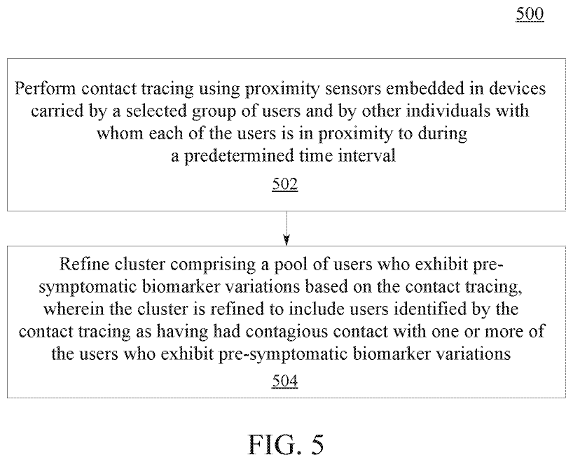

FIG. 5 is a flowchart of example method 500 of contact tracing in accordance with one or more embodiments. Method 500 can be performed by a system the same or similar to the ones described with reference to FIGS. 1 and 2. The system at block 502 performs contact tracing using proximity sensors embedded in devices carried by a selected group of users and by other individuals with whom each selected user is in proximity to during a predetermined time interval. The selected group of users can comprise users who exhibit pre-symptomatic biomarker variations associated with an infectious respiratory disease. Users who exhibit pre-symptomatic biomarker variations associated with an infectious respiratory disease can be identified by extracting biomarkers from sensor-detected signals with the portable devices of the users, as described above. Based on the biomarkers, the users can be identified from clusters generated and conditioned according to the procedures also described above.

At block 504, the system refines a cluster comprising a pool of users who exhibit pre-symptomatic biomarker variations based on the contact tracing. The cluster can be refined to include users identified by the contact tracing as having had contagious contact with one or more of the users who, as identified by a conditioned cluster, exhibit pre-symptomatic biomarker variations.

FIG. 6 illustrates a schematic of example computing node 600. In one or more embodiments, computing node 600 is an example of a suitable cloud computing node. Computing node 600 is not intended to suggest any limitation as to the scope of use or functionality of embodiments of the invention described herein. Computing node 600 is capable of performing any of the functionality described herein.

Computing node 600 includes a computer system 612, which is operational with numerous other general-purpose or special-purpose computing system environments or configurations. Examples of well-known computing systems, environments, and/or configurations that may be suitable for use with computer system 612 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 612 may be described in the general context of computer system-executable instructions, such as program modules, being executed by a computer system. Generally, program modules may include routines, programs, objects, components, logic, data structures, and so on that perform particular tasks or implement particular abstract data types. Computer system 612 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system 612 is shown in the form of a general-purpose computing device. The components of computer system 612 may include, but are not limited to, one or more processors 616, a memory 628, and a bus 618 that couples various system components including memory 628 to processor 616. As defined herein, “processor” means at least one hardware circuit configured to carry out instructions. The hardware circuit may be an integrated circuit. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

The carrying out of instructions of a computer program by a processor comprises executing the program. As defined herein, “execute” comprise a series of actions or events performed by the processor in accordance with one or more machine-readable instructions. “Executing,” as defined herein refers to the active performing of actions or events by the processor. The terms execute and executing are used synonymously herein.

Bus 618 represents one or more of any of several types of bus structures, including a memory bus or memory controller, a peripheral bus, an accelerated graphics port, and a processor or local bus using any of a variety of bus architectures. By way of example only, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, and PCI Express (PCIe) bus.

Computer system 612 typically includes a variety of computer system-readable media. Such media may be any available media that is accessible by computer system 612, and may include both volatile and non-volatile media, removable and non-removable media.

Memory 628 may include computer system readable media in the form of volatile memory, such as random-access memory (RAM) 630 and/or cache memory 632. Computer system 612 may further include other removable/non-removable, volatile/non-volatile computer system storage media. By way of example, storage system 634 can be provided for reading from and writing to a non-removable, non-volatile magnetic media and/or solid-state drive(s) (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 618 by one or more data media interfaces. As will be further depicted and described below, memory 628 may include at least one program product having a set (e.g., at least one) of program modules that are configured to carry out the functions of embodiments of the invention.

Program/utility 640, having a set (at least one) of program modules 642, may be stored in memory 628 by way of example, and not limitation, as well as an operating system, one or more application programs, other program modules, and program data. Each of the operating system, one or more application programs, other program modules, and program data or some combination thereof, may include an implementation of a networking environment. Program modules 642 generally carry out the functions and/or methodologies of embodiments of the invention as described herein. For example, one or more of the program modules may include IRDDS 200 or portions thereof.

Program/utility 640 is executable by processor 616. Program/utility 640 and any data items used, generated, and/or operated upon by computer system 612 are functional data structures that impart functionality when employed by computer system 612. As defined within this disclosure, a “data structure” is a physical implementation of a data model's organization of data within a physical memory. As such, a data structure is formed of specific electrical or magnetic structural elements in a memory. A data structure imposes physical organization on the data stored in the memory as used by an application program executed using a processor.

Computer system 612 may also communicate with one or more external devices 614 such as a keyboard, a pointing device, a display 624, etc.; one or more devices that enable a user to interact with computer system 612; and/or any devices (e.g., network card, modem, etc.) that enable computer system 612 to communicate with one or more other computing devices. Such communication can occur via input/output (I/O) interfaces 622. Still, computer system 612 can communicate with one or more networks such as a local area network (LAN), a general wide area network (WAN), and/or a public network (e.g., the Internet) via network adapter 620. As depicted, network adapter 620 communicates with the other components of computer system 612 via bus 618. It should be understood that although not shown, other hardware and/or software components could be used in conjunction with computer system 612. Examples, include, but are not limited to: microcode, device drivers, redundant processing units, external disk drive arrays, RAID systems, tape drives, and data archival storage systems, etc.

While computing node 600 is used to illustrate an example of a cloud computing node, it should be appreciated that a computer system using an architecture the same as or similar to that described in connection with FIG. 6 may be used in a non-cloud computing implementation to perform the various operations described herein. In this regard, the example embodiments described herein are not intended to be limited to a cloud computing environment. Computing node 600 is an example of a data processing system. As defined herein, “data processing system” means one or more hardware systems configured to process data, each hardware system including at least one processor programmed to initiate operations and memory.

Computing node 600 is an example of computer hardware. Computing node 600 may include fewer components than shown or additional components not illustrated in FIG. 6 depending upon the particular type of device and/or system that is implemented. The particular operating system and/or application(s) included may vary according to device and/or system type as may the types of I/O devices included. Further, one or more of the illustrative components may be incorporated into, or otherwise form a portion of, another component. For example, a processor may include at least some memory.

Computing node 600 is also an example of a server. As defined herein, “server” means a data processing system configured to share services with one or more other data processing systems. As defined herein, “client device” means a data processing system that requests shared services from a server, and with which a user directly interacts. Examples of a client device include, but are not limited to, a workstation, a desktop computer, a computer terminal, a mobile computer, a laptop computer, a netbook computer, a tablet computer, a smart phone, a personal digital assistant, a smart watch, smart glasses, a gaming device, a set-top box, a smart television and the like. In one or more embodiments, the various user devices described herein may be client devices. Network infrastructure, such as routers, firewalls, switches, access points and the like, are not client devices as the term “client device” is defined herein.

FIG. 7 illustrates an example portable device 700 in accordance with one or more embodiments described within this disclosure. Portable device 700 can include a memory 702, one or more processors 704 (e.g., image processors, digital signal processors, data processors), and interface circuitry 706.

In one aspect, memory 702, processor(s) 704, and/or interface circuitry 706 are implemented as separate components. In another aspect, memory 702, processor(s) 704, and/or interface circuitry 706 are integrated in one or more integrated circuits. The various components of portable device 700 can be coupled, for example, by one or more communication buses or signal lines (e.g., interconnects and/or wires). In one aspect, memory 702 may be coupled to interface circuitry 706 via a memory interface (not shown).

Sensors, devices, subsystems, and/or input/output (I/O) devices can be coupled to interface circuitry 706 to facilitate the functions and/or operations described herein, including the generation of sensor data. The various sensors, devices, subsystems, and/or I/O devices may be coupled to interface circuitry 706 directly or through one or more intervening I/O controllers (not shown).

For example, location sensor 710, light sensor 712, and proximity sensor 714 can be coupled to interface circuitry 706 to facilitate orientation, lighting, and proximity functions, respectively, of portable device 700. Location sensor 710 (e.g., a GPS receiver and/or processor) can be connected to interface circuitry 706 to provide geo-positioning sensor data. Electronic magnetometer 718 (e.g., an integrated circuit chip) can be connected to interface circuitry 706 to provide sensor data that can be used to determine the direction of magnetic North for purposes of directional navigation. Accelerometer 720 can be connected to interface circuitry 706 to provide sensor data that can be used to determine change of speed and direction of movement of a device in three dimensions. PPG sensor 722 (e.g., an integrated circuit) can be connected to interface circuitry 706 to detect volumetric changes in blood in a user's peripheral circulation. Heart rate sensor 724 can be connected to interface circuitry 706 to generate sensor data and facilitate measurement of a heartbeat and the determination of a heart rate.

Camera subsystem 726 can be coupled to an optical sensor 728. Optical sensor 728 can be implemented using any of a variety of technologies. Examples of optical sensor 728 include a charged coupled device (CCD), a complementary metal-oxide semiconductor (CMOS) optical sensor, and the like. Camera subsystem 726 and optical sensor 728 can be used to facilitate camera functions, such as recording images and/or video clips (hereafter “image data”). In one aspect, image data is a subset of sensor data.

Communication functions can be facilitated through one or more wireless communication subsystems 730. Wireless communications subsystem(s) 730 can include radio frequency receivers and transmitters, optical (e.g., infrared) receivers and transmitters, and so forth. The specific design and implementation of wireless communication subsystem(s) 730 can depend on the specific type of portable device 700 implemented and/or the communication network(s) over which portable device 700 is intended to operate.

For purposes of illustration, wireless communication subsystem(s) 730 may be designed to operate over one or more mobile networks (e.g., GSM, GPRS, EDGE), a Wi-Fi network that may include a WiMax network, a short-range wireless network (e.g., a Bluetooth network), and/or any combination of the foregoing. Wireless communication subsystem(s) 730 can implement hosting protocols such that portable device 700 can be configured as a base station for other wireless devices.

Audio subsystem 732 can be coupled to a speaker 734 and a microphone 736 to facilitate voice-enabled functions, such as voice recognition, voice replication, digital recording, audio processing, and telephony functions. Audio subsystem 732 is able to generate audio type sensor data. In one or more embodiments, microphone 736 may be utilized as a respirator sensor.

I/O devices 738 can be coupled to interface circuitry 706. Examples of I/O devices 738 include, for example, display devices, touch-sensitive display devices, track pads, keyboards, pointing devices, communication ports (e.g., USB ports), network adapters, buttons or other physical controls, and so forth. A touch-sensitive device such as a display screen and/or a pad is configured to detect contact, movement, breaks in contact, and the like using any of a variety of touch sensitivity technologies. Example touch-sensitive technologies include, for example, capacitive, resistive, infrared, and surface acoustic wave technologies, other proximity sensor arrays or other elements for determining one or more points of contact with a touch-sensitive device, and the like. One or more of I/O devices 738 may be adapted to control functions of sensors, subsystems, and such of portable device 700.

Portable device 700 further includes a power source 740. Power source 740 able to provide electrical power to various elements of portable device 700. In one embodiment, power source 740 is implemented as one or more batteries. The batteries may be implemented using any of a variety of different battery technologies, whether disposable (e.g., replaceable) or rechargeable. In another embodiment, power source 740 is configured to obtain electrical power from an external source and provide power (e.g., DC power) to the elements of portable device 700. In the case of a rechargeable battery, power source 740 further may include circuitry that is able to charge the battery or batteries when coupled to an external power source.

Memory 702 can include random access memory (e.g., volatile memory) and/or non-volatile memory, such as one or more magnetic disk storage devices, one or more optical storage devices, flash memory, and so forth. Memory 702 can store operating system 752, such as LINUX, UNIX, a mobile operating system, an embedded operating system, and the like. Operating system 752 may include instructions for handling system services and for performing hardware-dependent tasks.

Memory 702 may store additional program code 754. Examples of other program code 754 may include instructions to facilitate communicating with one or more additional devices, one or more computers, and/or one or more servers; graphic user interface processing; processing instructions to facilitate sensor-related functions; phone-related functions; electronic messaging-related functions; Web browsing-related functions; media processing-related functions; GPS and navigation-related functions; security functions; camera-related functions, including Web camera and/or Web video functions; and so forth. Memory 702 may also store one or more other applications 762.

Memory 702 may store IRDDS program code 756. IRDDS program code 756 can be adapted to implement the various embodiments of an IRDDS as described herein. Executing on one or more processors 704, IRDDS program code 756 performs signal processing and other operations for estimating a user's respiratory condition based on biomarkers extracted from signals captured by portable device 700. In one or more embodiments, IRDDS program code 756 facilitates the extraction of biomarkers from signals captured by portable device 700 (e.g., via a MEMS sensor, IMU, microphone 736 and/or other motion or sound transducers). The biomarkers correspond to one or more lung conditions and can be input to a machine learning classification model that may be incorporated into IRDDS program code 756 or accessible to the IRDDS via wireless communication subsystems 730 or a wired network connection of portable device 700. The IRDDS program code 756, using the machine learning classification model, can generate one or more estimated lung conditions of a user based on the extracted biomarkers. The biomarkers correspond to lung functions and include respiratory (or breathing) rate, respiratory or breathing cycle, fractional inspiratory time, I:E ratio, FEV1, FVC, and FEV1/FVC ratio. The biomarkers can be used for the identification and determination of lung-related problems and treatment of various diseases. Further aspects of operations performed through execution of IRDDS program code 756 are described herein with reference to the other figures.

Memory 702 may also store various types of data (not shown) such as sensor data, baseline data, data obtained by way of received user input(s), and/or data obtained by way of querying one or more external data sources.

The various types of instructions and/or program code described are provided for purposes of illustration and not limitation. The program code may be implemented as separate software programs, procedures, or modules. Memory 702 can include additional instructions or fewer instructions. Moreover, various functions of portable device 700 may be implemented in hardware and/or software, including in one or more signal processing and/or application-specific integrated circuits.

Program code stored within memory 702 and any data used, generated, and/or operated on by portable device 700 are functional data structures that impart functionality to a device when employed as part of the device. Further examples of functional data structures include, for example, sensor data, data obtained via user input, data obtained via querying external data sources, baseline information, and so forth.

In certain embodiments, one or more of the various sensors and/or subsystems described with reference to portable device 700 may be separate devices that are coupled or communicatively linked to portable device 700 through wired or wireless connections. For example, one or more (or all) of location sensor 710, light sensor 712, proximity sensor 714, gyroscope 716, magnetometer 718, accelerometer 720, altimeter 722, heart rate sensor 724, camera subsystem 726, audio subsystem 732, and so forth may be implemented as separate systems or subsystems that operatively couple to portable device 700 by way of I/O devices 738 and/or wireless communication subsystem(s) 730.

One or more of the sensors may be worn directly by the user and provide data to portable device 700 via a wired or wireless connection. Examples of additional sensors that are not illustrated in FIG. 7, but which may be used and/or worn by a user to provide sensor data to portable device 700 can include, for example, electrocardiography (ECG) sensors, photoplethysmography (PPG) sensors, gyroscopes, respiratory sensors, galvanic skin response (GSR) sensors, and the like. These additional sensors are represented in FIG. 7 by “other sensors” block 770. In one or more embodiments, sensors and/or subsystems as described herein are configured to generate sensor data that is stored in a memory external to portable device 700. Portable device 700 (e.g., processor(s) 704) may access externally stored sensor data for use and/or analysis as described herein.

Portable device 700 can include fewer components than those shown or include additional components other than those shown in FIG. 7 depending on the specific type of system that is implemented. Additionally, the particular operating system and/or application(s) and/or other program code included may also vary according to system type. Moreover, one or more of the illustrative components can be incorporated into, or otherwise form a portion of, another component. For example, a processor may include at least some memory.

Portable device 700 is provided for purposes of illustration and not limitation. A device and/or system configured to perform the operations described herein may have a different architecture than illustrated in FIG. 7. The architecture may be a simplified version of portable device 700 and may include a processor and memory storing instructions. The architecture may include one or more sensors as described herein. Portable device 700, or a similar system, can collect data using the various sensors of the device or sensors coupled thereto. It should be appreciated, however, that portable device 700 may include fewer sensors or other additional sensors. With this disclosure, data generated by a sensor is referred to as “sensor data.”

Example implementations of portable device 700 include, for example, a smartphone or other mobile device or phone, a wearable computing device (e.g., smartwatch), a dedicated medical device or other suitable handheld, wearable, or comfortably carriable electronic device, capable of sensing and processing sensor-detected signals and data. It will be appreciated that embodiments can be deployed as a standalone device or deployed as multiple devices in a distributed client-server networked system. For example, in certain embodiments, a smartwatch can operatively couple to a mobile device (e.g., smartphone). The mobile device may or may not be configured to interact with a remote server and/or computer system.

The terminology used herein is only for the purpose of describing particular embodiments and is not intended to be limiting. Notwithstanding, several definitions that apply throughout this document are now presented.

As defined herein, the singular forms of terms “a,” “an,” and “the” are intended to include the plural forms as well, unless the context clearly indicates otherwise.

As defined herein, “another” means at least a second or more.

As defined herein, “at least one,” “one or more,” and “and/or,” are open-ended expressions that are both conjunctive and disjunctive in operation unless explicitly stated otherwise. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, “automatically” means without user intervention.

As defined herein, “includes,” “including,” “comprises,” and/or “comprising,” specify the presence of stated features, integers, steps, operations, elements, and/or components, but do not preclude the presence or addition of one or more other features, integers, steps, operations, elements, components, and/or groups thereof.

As defined herein, “if” means “when” or “upon” or “in response to” or “responsive to,” depending upon the context. Thus, the phrase “if it is determined” or “if [a stated condition or event] is detected” may be construed to mean “upon determining” or “in response to determining” or “upon detecting [the stated condition or event]” or “in response to detecting [the stated condition or event]” or “responsive to detecting [the stated condition or event]” depending on the context.

As defined herein, “one embodiment,” “an embodiment,” “in one or more embodiments,” “in particular embodiments,” or similar language mean that a particular feature, structure, or characteristic described in connection with the embodiment is included in at least one embodiment described within this disclosure. Thus, appearances of the aforementioned phrases and/or similar language throughout this disclosure may, but do not necessarily, all refer to the same embodiment.

As defined herein, “in response to” and “responsive to” mean responding or reacting readily to an action or event. Thus, if a second action is performed “in response to” or “responsive to” a first action, there is a causal relationship between an occurrence of the first action and an occurrence of the second action. The terms “in response to” and “responsive to” indicate the causal relationship.

As defined herein, “real time” means a level of processing responsiveness that a user or system senses as sufficiently immediate for a particular process or determination to be made, or that enables the processor to keep up with some external process.

As defined herein, “substantially” means that the recited characteristic, parameter, or value need not be achieved exactly, but that deviations or variations, including for example, tolerances, measurement error, measurement accuracy limitations, and other factors known to those of skill in the art, may occur in amounts that do not preclude the effect the characteristic was intended to provide.

As defined herein, the terms “user,” “individual,” and “subject” mean a human being.

The terms first, second, etc. may be used herein to describe various elements. These elements should not be limited by these terms, as these terms are only used to distinguish one element from another unless stated otherwise or the context clearly indicates otherwise.

Various embodiments of the inventive aspects disclosed herein may be implemented in a system, as a method, and/or in a computer program product at any possible technical detail level of integration. The computer program product may include a computer readable storage medium (or media) having computer readable program instructions thereon for causing a processor to carry out aspects of the embodiments disclosed herein. As defined herein, “computer readable storage medium” means a storage medium that contains or stores program code for use by or in connection with an instruction execution system, apparatus, or device. “Computer readable storage medium,” as defined herein, is not a transitory, propagating signal per se. A computer readable storage medium may be, but is not limited to, an electronic storage device, a magnetic storage device, an optical storage device, an electromagnetic storage device, a semiconductor storage device, or any suitable combination of the foregoing. Memory elements, as described herein, are examples of a computer readable storage medium. A non-exhaustive list of more specific examples of a computer readable storage medium may include: a portable computer diskette, a hard disk, a random-access memory (RAM), a read-only memory (ROM), an erasable programmable read-only memory (EPROM or Flash memory), a static random-access memory (SRAM), a digital versatile disk (DVD), a memory stick, a floppy disk, or the like.

“Program code” is used interchangeably with “computer readable program instructions” within this disclosure. Computer readable program instructions described herein may be downloaded to respective computing/processing devices from a computer readable storage medium or to an external computer or external storage device via a network, such as the Internet, a LAN, a WAN, and/or a wireless network. The network may include copper transmission cables, optical transmission fibers, wireless transmissions, routers, firewalls, switches, gateway computers, and/or edge devices including edge servers. A network adapter cord or network interface in each computing/processing device receives computer readable program instructions from the network and forwards the computer readable program instructions for storage in a computer readable storage medium with the respective computing/processing device.

Computer readable program instructions for carrying out operations of the inventive arrangements disclosed herein may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language and/or procedural programming languages. The computer readable program instructions may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some embodiments, electronic circuitry including, for example, programmable logic circuitry, an FPGA, or a PLA may execute the computer readable program instructions by utilizing state information of the computer readable program instructions to personalize the electronic circuitry, in order to perform aspects of the inventive arrangements described herein.

Certain aspects of the inventive arrangements are described herein with reference to flowchart illustrations and/or block diagrams of methods, apparatus (systems), and computer program products according to embodiments of the inventive arrangements. It will be understood that each block of the flowchart illustrations and/or block diagrams, and combinations of blocks in the flowchart illustrations and/or block diagrams, can be implemented by computer readable program instructions (e.g., program code).

These computer readable program instructions may be provided to a processor of a computer, special-purpose computer or other programmable data processing apparatus to produce a machine, such that the instructions, which execute via the processor of the computer or other programmable data processing apparatus, create means for implementing the functions/acts specified in the flowchart and/or block diagram block or blocks. Operatively coupling the processor to program code instructions transforms the machine of the processor into a special-purpose machine for carrying out the instructions of the program code. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the inventive arrangements described herein. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments provided herein have been presented for purposes of illustration and are not intended to be exhaustive or limited to the form and examples disclosed. The terminology used herein was chosen to explain the principles of the inventive arrangements, the practical application or technical improvement over technologies found in the marketplace, and/or to enable others of ordinary skill in the art understand the embodiments disclosed herein. Modifications and variations may be apparent to those of ordinary skill in the art without departing from the scope and spirit of the described inventive arrangements. Accordingly, reference should be made to the following claims, rather than the foregoing disclosure, as indicating the scope of such features and implementations.

What is claimed is:

1. A computer-implemented method within an infectious disease detection system, the computer-implemented method comprising:
monitoring during a predetermined time interval electronic communications generated by an ensemble of network-connected portable devices, each electronic communication corresponding to one of a plurality of users using a corresponding one of the portable devices;
processing the electronic communications by extracting from each electronic communication one or more biomarkers obtained by processing signals captured by a corresponding portable device, the signals generated in response to sensor-detected respiratory activity of a user of the corresponding portable device;
generating one or more clusters of users based on the one or more biomarkers corresponding to each of the users;
conditioning the one or more clusters based on pre-condition data extracted from the electronic communications, the pre-condition data corresponding to users who are determined to have a pre-existing health condition;
determining, based on context data extracted from the electronic communications, user co-locations and pre-symptomatic biomarker variation co-occurrences among a pool of users who exhibit pre-symptomatic biomarker variations associated with an infectious respiratory disease; and
predicting a location of a possible outbreak of the infectious respiratory disease based on the user co-locations and pre-symptomatic biomarker variation co-occurrences.

2. The method of claim 1, wherein each electronic communication monitored is an electronic communication conveyed over a communications network by a corresponding network-connected portable device in response to the corresponding portable device identifying a biomarker anomaly associated with the user of the corresponding device.

3. The method of claim 1, further comprising conveying an electronic notification via a network connection to a communication device of at least one individual, the electronic notification indicating the predicted location and a predicted timing of the possible outbreak of the infectious respiratory disease and likelihood of infection for an individual.

4. The method of claim 1, wherein the predicting comprises estimating one or more epidemiological model parameters based on the context data extracted from the electronic communications.

5. The method of claim 1, further comprising providing an electronic request to each of the pool of users who exhibit pre-symptomatic biomarker variations associated with an infectious respiratory disease, requesting each user who exhibits pre-symptomatic biomarker variations to perform one or more active tests using a portable device.

6. The method of claim 5, further comprising:
determining which users who exhibit pre-symptomatic biomarker variations exhibit one or more same symptoms based on the one or more active tests performed; and
refining the pool of users who exhibit pre-symptomatic biomarker variations based on the determining which users exhibit one or more same symptoms to include in the pool only users who exhibit one or more of the same systems, while excluding any other users.

7. The method of claim 1, further comprising:
performing contact tracing using proximity sensors embedded in devices carried by the users and by other individuals with whom the users are in proximity to during the predetermined time interval; and
refining the cluster comprising a pool of users who exhibit pre-symptomatic biomarker variations based on the contact tracing, wherein the refining comprises including in the pool users identified by contact tracing as having had contagious contact with one or more users who exhibit pre-symptomatic biomarker variations.

8. A system, comprising:
a processor configured to initiate operations including:
monitoring during a predetermined time interval electronic communications generated by an ensemble of network-connected portable devices, each electronic communication corresponding to one of a plurality of users using a corresponding one of the portable devices;
processing the electronic communications by extracting from each electronic communication one or more biomarkers obtained by processing signals captured by a corresponding portable device, the signals generated in response to sensor-detected respiratory activity of a user of the corresponding portable device;
generating one or more clusters of users based on the one or more biomarkers corresponding to each of the users;
conditioning the one or more clusters based on pre-condition data extracted from the electronic communications, the pre-condition data corresponding to users who are determined to have a pre-existing health condition;
determining, based on context data extracted from the electronic communications, user co-locations and pre-symptomatic biomarker variation co-occurrences among a pool of users who exhibit pre-symptomatic biomarker variations associated with an infectious respiratory disease; and
predicting a location of a possible outbreak of the infectious respiratory disease and likelihood of infection for individuals based on the user co-locations and pre-symptomatic biomarker variation co-occurrences.

9. The system of claim 8, wherein each electronic communication monitored is an electronic communication conveyed over a communications network by a corresponding network-connected portable device in response to the corresponding portable device identifying a biomarker anomaly associated with the user of the corresponding device.

10. The system of claim 8, wherein the processor is configured to initiate operations further comprising conveying an electronic notification via a network connection to a communication device of at least one individual, the electronic notification indicating the predicted location and a predicted timing of the possible outbreak of the infectious respiratory disease and likelihood of infection for an individual.

11. The system of claim 8, wherein the predicting comprises estimating one or more epidemiological model parameters based on the context data extracted from the electronic communications.

12. The system of claim 8, wherein the processor is configured to initiate operations further comprising providing an electronic request to each of the pool of users who exhibit pre-symptomatic biomarker variations associated with an infectious respiratory disease, requesting each user who exhibits pre-symptomatic biomarker variations to perform one or more active tests using a portable device.

13. The system of claim 12, wherein the processor is configured to initiate operations further comprising:
determining which users who exhibit pre-symptomatic biomarker variations exhibit one or more same symptoms based on the one or more active tests performed; and
refining the pool of users who exhibit pre-symptomatic biomarker variations based on the determining which users exhibit one or more same symptoms to include in the pool only users who exhibit one or more of the same systems, while excluding any other users.

14. A computer program product comprising one or more computer readable storage media having program instructions collectively stored therein, the program instructions executable by a processor to cause the processor to initiate operations comprising:
monitoring during a predetermined time interval electronic communications generated by an ensemble of network-connected portable devices, each electronic communication corresponding to one of a plurality of users using a corresponding one of the portable devices;
processing the electronic communications by extracting from each electronic communication one or more biomarkers obtained by processing signals captured by a corresponding portable device, the signals generated in response to sensor-detected respiratory activity of a user of the corresponding portable device;
generating one or more clusters of users based on the one or more biomarkers corresponding to each of the users;
conditioning the one or more clusters based on pre-condition data extracted from the electronic communications, the pre-condition data corresponding to users who are determined to have a pre-existing health condition;
determining, based on context data extracted from the electronic communications, user co-locations and pre-symptomatic biomarker variation co-occurrences among a pool of users who exhibit pre-symptomatic biomarker variations associated with an infectious respiratory disease; and
predicting a location of a possible outbreak of the infectious respiratory disease based on the user co-locations and pre-symptomatic biomarker variation co-occurrences.

15. The computer program product of claim 14, wherein each electronic communication monitored is an electronic communication conveyed over a communications network by a corresponding network-connected portable device in response to the corresponding portable device identifying a biomarker anomaly associated with the user of the corresponding device.

16. The computer program product of claim 15, wherein the program instructions are executable by the processor to cause the processor to initiate operations further comprising conveying an electronic notification via a network connection to a communication device of at least one individual, the electronic notification indicating the predicted location and a predicted timing of the possible outbreak of the infectious respiratory disease and likelihood of infection for an individual.

17. The computer program product of claim 14, wherein the predicting comprises estimating one or more epidemiological model parameters based on the context data extracted from the electronic communications.

18. The computer program product of claim 14, wherein the program instructions are executable by the processor to cause the processor to initiate operations further comprising providing an electronic request to each of the pool of users who exhibit pre-symptomatic biomarker variations associated with an infectious respiratory disease, requesting each user who exhibits pre-symptomatic biomarker variations to perform one or more active tests using a portable device.

19. The computer program product of claim 18, wherein the program instructions are executable by the processor to cause the processor to initiate operations further comprising:
determining which users who exhibit pre-symptomatic biomarker variations exhibit one or more same symptoms based on the one or more active tests performed; and
refining the pool of users who exhibit pre-symptomatic biomarker variations based on the determining which users exhibit one or more same symptoms to include in the pool only users who exhibit one or more of the same systems, while excluding any other users.

20. The computer program product of claim 14, wherein the program instructions are executable by the processor to cause the processor to initiate operations further comprising:
performing contact tracing using proximity sensors embedded in devices carried by the users and by other individuals with whom the users are in proximity to during the predetermined time interval; and
refining the cluster comprising a pool of users who exhibit pre-symptomatic biomarker variations based on the contact tracing, wherein the refining comprises including in the pool users identified by contact tracing as having had contagious contact with one or more users who exhibit pre-symptomatic biomarker variations.
